(19) United States
(12) Patent Application Publication
(10) Pub. No.: US 2023/0405167 A1
(43) Pub. Date: Dec. 21, 2023

(54) APPARATUS FOR THE TREATMENT OF A BIOLOGICAL SURFACE USING ATMOSPHERIC- PRESSURE PLASMA

(71) Applicant: L'Oreal, Paris (FR)

(72) Inventors: Grégoire CHARRAUD, Clichy (FR); Matthieu JACOB, Clichy (FR); Thibault LESTAVEL, Paris (FR); Sylvain ROULAND, Coulommiers (FR); Nathan OLFF, Villeurbanne (FR)

(73) Assignee: L'Oreal, Paris (FR)

(21) Appl. No.: 18/246,496

(22) PCT Filed: Sep. 6, 2021
(86) PCT No.: PCT/EP2021/074476

§ 371 Date: Mar. 23, 2023

(30) Foreign Application Priority Data
Sep. 23, 2020 (FR) 2009661

(51) Int. Cl. A61L 2/14; H05H 1/34

(52) U.S. Cl. CPC A61L 2/14; H05H 2245/34; H05H 1/3473

(57) ABSTRACT

The invention relates to an apparatus for the treatment of a biological surface using atmospheric-pressure plasma, comprising: —a device for generating atmospheric-pressure plasma (DGP), and —a control device (DC) configured to control an amount of plasma generated by said plasma generation device (DGP) within a given time period and to limit generation of the plasma by said plasma generation device (DGP) so that the amount of plasma generated over the given time period does not exceed a maximum threshold.

The invention notably relates to controlling the generation of atmospheric-pressure plasma, in particular to an apparatus for the treatment of a biological surface using atmospheric-pressure plasma and to adjusting an amount of plasma generated by such an apparatus.

Atmospheric-pressure plasma (also called “cold plasma” or “non-thermal plasma”) can interact with biological tissues and cells during a cosmetic treatment.

Among the possible applications, atmospheric-pressure plasma can be used in biology and in medicine for sterilization, disinfection, decontamination and wound healing.

Thus, apparatuses for the treatment of a biological surface using atmospheric plasma which are configured to address these various applications are known.

In particular, some of these apparatuses are configured for the cosmetic treatment of the skin using atmospheric-pressure plasma.

Such apparatuses are therefore configured to apply a plasma to the surface of the skin, and in particular to the surface of the face, notably for cosmetic treatment of acne.

The US patent application published under the number US 2020/0038673 describes such a cosmetic treatment apparatus.

Additionally, the generation of atmospheric-pressure plasma can produce ozone.

Since atmospheric-pressure plasma may be generated close to a user's face, ozone may be inhaled through this user's nose or mouth.

However, ozone is a potentially toxic gas when large amounts are breathed in. In particular, ozone can be extremely harmful for the lungs, the kidneys, the brain and the eyes.

By way of example, ozone can lead to pulmonary oedema, coughing, problems with vision and renal and neurological difficulties.

However, some of these known apparatuses have the drawback of being able to produce an amount of ozone that may be harmful for the user when they are used to generate atmospheric-pressure plasma over a long period.

The invention aims to remedy this drawback.

One subject of the invention is an apparatus for the treatment of a biological surface, notably for the cosmetic treatment of the skin, using atmospheric-pressure plasma, comprising:
a device for generating atmospheric-pressure plasma, and
a control device configured to control an amount of plasma generated by said plasma generation device within a given time period and to limit generation of the plasma by said plasma generation device so that the amount of plasma generated over the given time period does not exceed a maximum threshold.

In particular, the maximum threshold corresponds to a threshold lower than or equal to a maximum amount of plasma for which the amount of ozone that may be produced during the generation of such a maximum amount of plasma is a maximum amount of ozone to which a human being may be exposed over said time period without harming them.

Such an apparatus is therefore configured to prevent a user using this apparatus from being exposed to an amount of ozone that may be harmful for this user.

In one advantageous embodiment, the control device is configured to prevent the generation of plasma by the plasma generation device when the amount of plasma generated over the given time period reaches the maximum threshold.

Once the given time period has ended, the control device is configured to be able to authorize the generation of plasma again.

In one advantageous embodiment, the control device is configured to limit the amount of plasma that can be generated instantaneously by the plasma generation device when the amount of plasma generated over the given time period reaches an intermediate threshold below the maximum threshold.

By limiting the amount of plasma generated instantaneously when the amount of plasma generated over the given time period is higher than the intermediate threshold, it is possible to extend the duration for which plasma may be generated before reaching the maximum threshold.

In one advantageous embodiment, the intermediate threshold is between 70% and 90% of the maximum threshold, preferably of the order of 80% of the maximum threshold.

In one advantageous embodiment, the maximum threshold is defined as being equal to 0.1 ppm ozone.

In one advantageous embodiment, the plasma generation device comprises an electrical power source configured to deliver a supply current, and a plasma generator configured to generate plasma using said supply current. The control device is then configured to monitor the supply current delivered by said power source to control the amount of plasma generated by the plasma generator.

Advantageously, the plasma generator is configured to be able to generate plasma when the supply current is higher than a given value, referred to as the plasma generation threshold value.

In one advantageous embodiment, the control device comprises:
a comparator configured to compare the value of the supply current with said plasma generation threshold value,
a counter configured to count, over the given time period, a duration, referred to as the duration of exposure, for which the value of the supply current is higher than the plasma generation threshold value, and
a control circuit configured to limit the supply current according to the duration counted by the counter over the given time period, so that the amount of plasma generated over the given time period does not exceed a maximum threshold.

In one advantageous embodiment, the control circuit is configured to prevent the power source from delivering a supply current to the plasma generator when the duration counted by the counter reaches a maximum duration.

In one advantageous embodiment, the control circuit is configured to limit the supply current delivered by the power source to the plasma generator when the duration counted by the counter reaches an intermediate duration shorter than the maximum duration.

In one advantageous embodiment, the time period is one day.

In one advantageous embodiment, the apparatus further comprises an alert device configured to issue an alert signal when the amount of plasma generated over the given time period reaches the maximum threshold.

Preferably, the apparatus further comprises:
a memory configured to be able to store at least one user identifier, and
a user selection device configured to select a user identifier from among said at least one user identifier stored in said memory.

The control device is then configured to control, for each user, an amount of plasma generated by said plasma generation device within a given time period and to limit generation of the plasma by said plasma generation device so that the amount of plasma generated over the given time period does not exceed a maximum threshold for each user.

In one advantageous embodiment, the counter is configured to count a duration of exposure for each user, the duration of exposure associated with a user being counted when the value of the supply current is higher than the plasma generation threshold value and when the identifier of this user is selected by the device for selecting this user.

Another subject of the invention is a method for adjusting an amount of plasma generated by a device for generating atmospheric-pressure plasma, in particular for treatment of a biological surface using atmospheric-pressure plasma, comprising:
controlling the amount of plasma generated by the plasma generation device within a given time period, and
limiting the generation of the plasma by said plasma generation device so that the amount of plasma generated over the given time period does not exceed a maximum threshold.

Other advantages and features of the invention will become apparent from examining the detailed description of implementations of the invention, which are in no way limiting, and the appended drawings, in which:

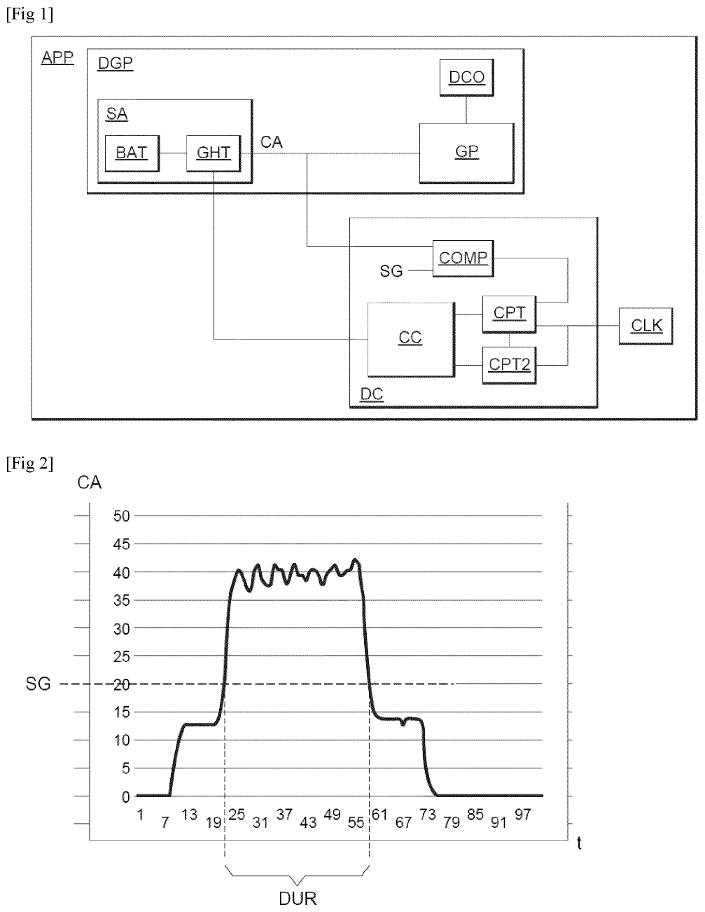

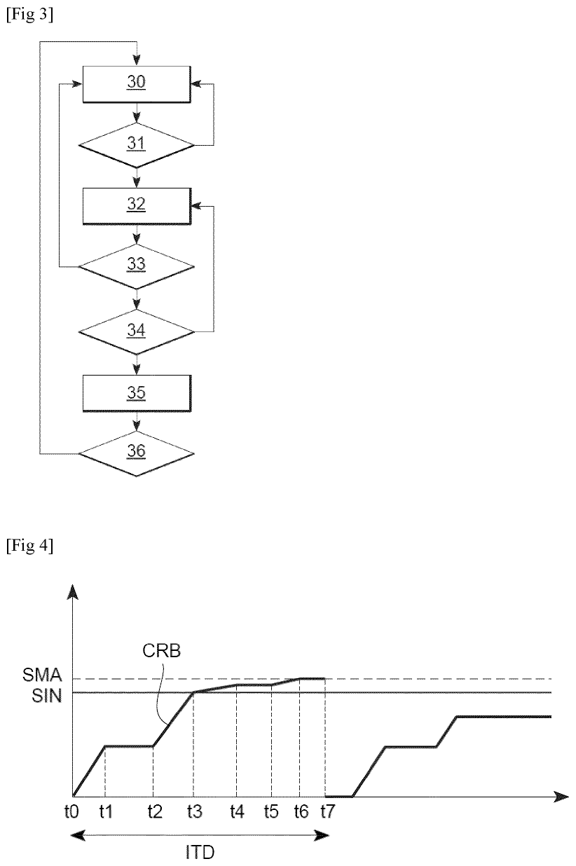

FIG. 1, FIG. 2, FIG. 3 and FIG. 4 schematically illustrate embodiments and implementations of the invention.

FIG. 1 illustrates an apparatus APP for the treatment of a biological surface, notably for the cosmetic treatment of the skin, using atmospheric-pressure plasma according to one embodiment of the invention.

Preferably, the apparatus APP is sized so as to be able to be held in just one of a user's hands. The apparatus APP may, for example, be similar in structure to the apparatus described in the US patent application published under the number US 2020/0038673.

The user is generally a person using the apparatus for themself. However, the user of the apparatus may also be a person using the apparatus to treat another person. For the sake of simplicity, the term user will refer to the person undergoing the treatment.

The apparatus APP comprises a plasma generation device DGP.

The plasma generation device DGP comprises a power source SA.

The power source SA comprises a battery BAT and a high-voltage generator GHT electrically connected to said battery BAT.

The battery BAT is configured to store energy and to be charged with energy, for example from a power connector of the apparatus APP. The battery BAT allows the high-voltage signal generator GHT to be supplied with power.

The high-voltage signal generator GHT is configured to produce a supply current CA from the energy stored in the battery BAT.

In particular, the high-voltage signal generator GHT is configured to produce a sinusoidal voltage. This sinusoidal voltage is, for example, between +/−1 kV and +/−3 kV. Additionally, the frequency of this sinusoidal voltage is preferably between 20 kHz and 60 kHz.

The plasma generation device DGP further comprises a generator GP of atmospheric-pressure plasma. This plasma generator GP is connected at the output of the high-voltage signal generator GHT. This plasma generator GP is therefore configured to receive the supply current CA produced by the high-voltage signal generator GHT. This plasma generator GP is configured to be able to generate, outside the apparatus APP, an atmospheric-pressure plasma using the supply current that it receives.

The plasma generator GP conventionally comprises a live electrode and a ground electrode and a dielectric barrier placed against the live electrode. In one advantageous embodiment, the ground electrode is the biological surface to which the plasma generator is applied. This biological surface thus acts as a floating ground electrode. An atmospheric-pressure plasma can be generated between these two electrodes. In particular, when the live electrode is supplied with power with respect to the ground electrode by the power source, a plasma is generated from a gas located between the electrodes. In one advantageous embodiment, the gas is atmospheric air located between the dielectric barrier and the floating ground electrode (the biological surface treated). The gas may contain at least one noble gas such as helium, argon or neon.

In particular, the plasma generator GP is configured to be able to generate plasma when the supply current CA is higher than a given value, referred to as the plasma generation threshold value SG, as illustrated in FIG. 2.

In particular, FIG. 2 illustrates one implementation in which the supply current CA is higher than the plasma generation threshold value SG for a duration DUR. Plasma is thus generated for the duration DUR. The value of the supply current CA is shown on the y-axis in amps and time is shown on the x-axis in seconds.

The plasma generation device DGP further comprises a control device DCO configured to control the high-voltage signal generator GHT. In particular, the control device DCO may comprise at least one push-button that is accessible from outside the apparatus APP which allows the user to activate the high-voltage signal generator GHT.

The apparatus APP further comprises a control device DC configured to control an amount of plasma generated by the plasma generation device DGP within a given time period ITD.

This given time period ITD may, for example, be one day.

In particular, the control device DC comprises a comparator COMP. The comparator COMP is configured to compare the value of the supply current with the plasma generation threshold value SG. In particular, the comparator COMP may be a current controller.

The control device DC also comprises a counter CPT. The counter CPT is configured to count, over the given time period, a duration, referred to as the duration of exposure, for which the value of the supply current is higher than the plasma generation threshold value SG.

In particular, the counter CPT may be configured to count a number of rising and/or falling edges of a clock signal generated by a clock CLK for as long as the value of the supply current is higher than the plasma generation threshold value SG over the given time interval.

The counter CPT is configured to be able to be reset at the end of the given time period.

In particular, the control device DC may comprise a second counter CPT2 for measuring the given time period ITD. In particular, this second counter may be configured to count a number of rising and/or falling edges of the clock signal generated by the clock CLK. Thus, when the second counter CPT2 reaches a value representative of the end of the given time period, the second counter CPT2 is configured to issue a signal for resetting the counter CPT. The second counter CPT2 may also be reset once it reaches the value representative of the end of the given time period.

The control device DC further comprises a control circuit CC. The control circuit CC is configured to limit the supply current according to the duration counted by the counter CPT over the given time period, so that the amount of plasma generated over the given time period does not exceed a maximum threshold SMA.

The maximum threshold corresponds to a threshold lower than or equal to a maximum amount of plasma for which the amount of ozone that may be produced during the generation of such a maximum amount of plasma is a maximum amount of ozone to which a human being may be exposed over said time period without harming them.

For example, the maximum threshold may be defined as being equal to 0.1 ppm.

In particular, the control circuit CC may be configured to prevent the power source SA from delivering a supply current to the plasma generator GP when the duration of exposure counted by the counter CPT reaches a maximum duration, referred to as the maximum duration of exposure.

In this regard, it is possible, for example, to use a switch, for example a MOS transistor, which can be controlled over its gate via a control signal, to deactivate or otherwise the signal generator GHT, the control signal being delivered by a logic circuit.

The control device DC is thus configured to prevent the generation of plasma by the plasma generation device DGP when the amount of plasma generated over the given time period reaches the maximum threshold.

Additionally, once the given time period has ended, the control device is configured to be able to authorize the generation of plasma again.

Such an apparatus is therefore configured to prevent a user using this apparatus from being exposed to an amount of ozone that may be harmful for this user.

Furthermore, the control circuit CC may also be configured to limit the supply current delivered by the power source SA to the plasma generator GP when the duration of exposure counted by the counter CPT reaches an intermediate duration shorter than the maximum duration.

The control device DC is thus configured to limit the amount of plasma that can be generated instantaneously by the plasma generation device DGP when the amount of plasma generated over the given time period reaches an intermediate threshold SIN below the maximum threshold. The intermediate threshold may be between 70% and 90% of the maximum threshold, for example.

To limit the supply current delivered by the power source to the plasma generator GP, it is possible to lower the value of the high voltage delivered by the high voltage signal generator GHT.

As a variant, it is possible to configure the high-voltage signal generator so that it is able to generate a pulse-width modulation signal (PWM signal). Thus, to limit the supply power delivered to the plasma generation device, it is possible to decrease a duty cycle of this pulse-width modulation signal.

By limiting the amount of plasma that may be generated instantaneously when the amount of plasma generated over the given time period is higher than the intermediate threshold, it is possible to extend the duration for which plasma may be generated before reaching the maximum threshold SMA.

Once the given time period ITD has ended, the control device is configured to be able to lift the limitation on the amount of plasma that may be generated instantaneously.

The control device DC is also configured to be able to generate a signal, referred to as the alarm signal, when the amount of plasma generated over the given time period has reached the maximum threshold SMA.

Additionally, the apparatus further comprises an alert device DA. The alert device DA is configured to issue an alert signal when the amount of plasma generated over the given time period reaches the maximum threshold SMA.

In particular, the alert device DA is electrically connected to the control device so as to be able to receive the alarm signal generated by the control device DC.

The alert device may comprise a transmitter configured to transmit a radio signal to a receiver, for example a signal in accordance with the Bluetooth standard, when it receives the alarm signal. The receiver may, for example, be the user's smartphone. This smartphone then comprises software configured to process the radio signal received in order to inform the user, notably by means of a message which may be displayed on a screen of the smartphone, that the amount of plasma generated over the given time period has reached the maximum threshold SMA.

As a variant, the alert device DA may comprise a vibrator configured to vibrate the apparatus. The vibration that may be generated by the vibrator then serves as an alert signal to notify the user that the amount of plasma generated over the given time period has reached the maximum threshold SMA.

As a variant, the alert device DA may comprise a light-emitting diode arranged in the apparatus so as to be able to be visible from outside the apparatus. The light emitted by the light-emitting diode then serves as an alert signal to notify the user that the amount of plasma generated over the given time period has reached the maximum threshold SMA.

Additionally, the apparatus APP may be configured to be able to be used by multiple different users.

Thus, preferably, the apparatus further comprises a memory MEM configured to be able to store at least one identifier of the user.

The apparatus also comprises a user selection device DSP. The user selection device DSP is configured to select a user identifier from among said at least one user identifier stored in said memory.

In particular, the apparatus APP may comprise a microcontroller that may be used as a user selection device DSP.

The user may notably be selected using a push-button provided on the apparatus APP.

As a variant, the user selection device DSP may be configured to automatically detect the user. This detection may be achieved by identifying, for example via a signal in accordance with the Bluetooth standard, a MAC address of the smartphone of the user located close to the apparatus APP, who may also be the user of the apparatus APP.

The control device DC is then configured to control the amount of plasma generated by said plasma generation device DGP within the given time period for each user.

In particular, the counter CPT is configured to be able to count a duration of exposure within the given time period for each user.

More particularly, a duration of exposure is associated with each user identifier.

The duration of exposure associated with a user identifier is counted by virtue of the counter CPT when this user identifier is selected by the selection device.

The control circuit CC is configured to limit the supply current according to the duration of exposure counted for the selected user identifier within the given time period, so that the amount of plasma generated over the given time period does not exceed a maximum threshold for this user identifier.

The control device DC thus makes it possible to limit generation of the plasma by said plasma generation device DGP so that the amount of plasma generated over the given time period does not exceed a maximum threshold SMA for each user.

FIG. 3 illustrates one implementation of a method for adjusting an amount of plasma generated by a device for generating atmospheric-pressure plasma for treatment of a biological surface, notably cosmetic treatment of the skin, using atmospheric-pressure plasma. This adjustment method may be implemented by the apparatus APP described above.

This adjustment method makes it possible to prevent the amount of plasma generated by the plasma generation device from being higher than a maximum threshold SMA as described above.

In this adjustment method, the plasma generation device is first set in step 30 to be able to generate an instantaneous amount of plasma in normal operation. This instantaneous amount of plasma in normal operation is an amount of plasma defined by the manufacturer of the apparatus APP.

The adjustment method further comprises controlling the amount of plasma generated over a given time period, for example one day.

Control is implemented by the control device.

The amount of plasma generated over the given time period is controlled by controlling the duration of exposure measured by the counter CPT.

In particular, the counter CPT is used to measure the duration of exposure on the basis of the result of the comparison performed by the comparator between the value of the supply current supplying the plasma generator GP and the plasma generation threshold value SG. As seen above, the duration of exposure is the total duration for which the value of the supply current CA is higher than the plasma generation threshold value SG.

More particularly, in step 31, the control device determines whether the duration of exposure has reached said intermediate duration, which is shorter than the maximum duration of exposure. This intermediate duration is representative of an intermediate threshold for the amount of plasma that may be generated over the time period below the maximum threshold.

If the duration of exposure has not yet reached the intermediate duration, the control circuit CC does not limit the amount of plasma that may be generated instantaneously by the plasma generation device DGP. The amount of plasma that may be generated instantaneously by the plasma generation device therefore remains the instantaneous amount of plasma in normal operation set in step 30.

However, if the duration of exposure has reached the intermediate duration, then the control circuit CC limits, in step 32, the amount of plasma that may be generated instantaneously by the plasma generation device DGP.

In step 33, the control device further determines whether the second counter CPT2 has reached the end of the time period.

Thus, if the second counter CPT2 has reached the end of the time period, then the limitation on the amount of plasma that may be generated instantaneously by the plasma generation device is lifted, with a return to step 30. The plasma generation device may then once again instantaneously generate said instantaneous amount of plasma in normal operation.

However, if the second counter CPT2 has not yet reached the end of the time period, then the control device DC determines, in step 34, whether the amount of plasma generated over the given time period has reached the maximum duration of exposure.

If the duration of exposure measured by the counter CPT has not reached the maximum duration of exposure, then the control circuit CC continues to uphold the limitation on the amount of plasma that may be generated instantaneously by the plasma generation device DGP.

However, if the duration of exposure measured by the counter CPT has reached the maximum duration of exposure, then the control circuit CC prevents, in step 35, the generation of plasma by the plasma generation device DGP until the end of the given time period.

Thus, the control device continues to control, in step 36, the duration measured by the second counter CPT2 so as to determine whether it has reached the end of the given time period.

Once the duration measured by the second counter CPT2 has reached the end of the given time period, the control circuit CC once again allows the generation of plasma by the plasma generation device DGP, with a return to step 30. Thus, the plasma generation device DGP may once again instantaneously generate said instantaneous amount of plasma in normal operation.

An example of the result that may be obtained by implementing such a method is illustrated in FIG. 4. In this figure, the curve CRB shows the change over time in the amount of plasma generated over a time period which is equal to one day.

In particular, the user starts their treatment using the apparatus APP at a time t0. The plasma generation device DGP therefore generates plasma a first time from time t0 to a time t1. The plasma generation device DGP then instantaneously generates said instantaneous amount of plasma in normal operation.

Next, at a time t1, the user stops their treatment, and then resumes their treatment at a time t2.

Because the duration of exposure counted by the counter CNT has not yet reached the intermediate duration, the plasma generation device DGP may continue to instantaneously generate said instantaneous amount of plasma in normal operation. However, at time t3, the duration of exposure counted by the counter CNT reaches the intermediate duration. The amount of plasma generated over the given time period therefore reaches the intermediate threshold SIN. The control circuit then limits the amount of plasma that may be generated instantaneously by the plasma generation device DGP for this user. The plasma generation device DGP therefore continues to generate plasma, but the amount of plasma generated instantaneously thereby is limited.

Next, at a time t4, the user again stops their treatment, and then resumes their treatment at a time t5.

At time t5, because the duration of exposure counted by the counter CNT has not yet reached the maximum duration of exposure, the plasma generation device DGP may continue to instantaneously generate a limited amount of plasma.

However, at time t6, the duration of exposure counted by the counter CNT reaches the maximum duration of exposure. The amount of plasma generated over the given time period then reaches the maximum threshold SMA. The control circuit then prevents the generation of plasma by the plasma generation device DGP until the end of the given time period for this user.

At time t7, the given time period ITD ends and the user may again resume their treatment.

1. Apparatus for the treatment of a biological surface using atmospheric-pressure plasma, comprising:
a device for generating atmospheric-pressure plasma (DGP), and
a control device (DC) configured to control an amount of plasma generated by said plasma generation device (DGP) within a given time period and to limit generation of the plasma by said plasma generation device (DGP) so that the amount of plasma generated over the given time period does not exceed a maximum threshold.

2. Apparatus according to claim 1, in which the control device (DC) is configured to prevent the generation of plasma by the plasma generation device (DGP) when the amount of plasma generated over the given time period reaches the maximum threshold.

3. Apparatus according to claim 2, in which the control device (DC) is configured to limit the amount of plasma that can be generated instantaneously by the plasma generation device (DGP) when the amount of plasma generated over the given time period reaches an intermediate threshold below the maximum threshold.

4. Apparatus according to claim 3, in which the intermediate threshold is between 70% and 90% of the maximum threshold.

5. Apparatus according to claim 1, in which the maximum threshold is defined as being equal to 0.1 ppm ozone.

6. Apparatus according to claim 1, in which the plasma generation device (DGP) comprises an electrical power source (SA) configured to deliver a supply current, and a plasma generator (GP) configured to generate plasma using said supply current, and in which the control device (DC) is configured to monitor the supply current delivered by said power source to control the amount of plasma generated by the plasma generator (GP).

7. Apparatus according to claim 6, in which the plasma generator (GP) is configured to be able to generate plasma when the supply current is higher than a given value, referred to as the plasma generation threshold value, and in which the control device (DC) comprises:
a comparator (CMP) configured to compare the value of the supply current with said plasma generation threshold value,
a counter (CPT) configured to count, over the given time period, a duration, referred to as the duration of exposure, for which the value of the supply current is higher than the plasma generation threshold value, and
a control circuit (CC) configured to limit the supply current according to the duration counted by the counter (CPT) over the given time period, so that the amount of plasma generated over the given time period does not exceed a maximum threshold.

8. Apparatus according to claim 7, in which the control circuit (CC) is configured to prevent the power source (SA) from delivering a supply current to the plasma generator (GP) when the duration counted by the counter (CPT) reaches a maximum duration.

9. Apparatus according to claim 8, in which the control circuit (CC) is configured to limit the supply current delivered by the power source (SA) to the plasma generator (GP) when the duration counted by the counter (CPT) reaches an intermediate duration shorter than the maximum duration.

10. Apparatus according to claim 1, in which the time period is one day.

11. Apparatus according to claim 1, further comprising an alert device (DA) configured to issue an alert signal when the amount of plasma generated over the given time period reaches the maximum threshold.

12. Apparatus according to claim 1, further comprising:
a memory (MEM) configured to be able to store at least one user identifier, and
a user selection device (DSP) configured to select a user identifier from among said at least one user identifier stored in said memory (MEM),
and in which the control device (DC) is configured to control, for each user, an amount of plasma generated by said plasma generation device within a given time period and to limit generation of the plasma by said plasma generation device (DGP) so that the amount of plasma generated over the given time period does not exceed a maximum threshold for each user.

13. Apparatus according to claim 7, in which the counter (CPT) is configured to count a duration of exposure for each user, the duration of exposure associated with a user being counted when the value of the supply current is higher than the plasma generation threshold value and when the identifier of this user is selected by the user selection device (DSP).

14. Method for adjusting an amount of plasma generated by a device for generating atmospheric-pressure plasma (DGP), in particular for treatment of a biological surface using atmospheric-pressure plasma, comprising:
controlling the amount of plasma generated by the plasma generation device (DGP) within a given time period, and
limiting the generation of the plasma by said plasma generation device (DGP) so that the amount of plasma generated over the given time period does not exceed a maximum threshold.
